(19) United States
(12) Patent Application Publication
(10) Pub. No.: US 2026/0100253 A1
(43) Pub. Date: Apr. 9, 2026

(54) IMAGE PROCESSING DEVICE, IMAGE PROCESSING METHOD, AND NON-TRANSITORY COMPUTER READABLE MEDIUM

(71) Applicant: Preferred Networks, Inc., Tokyo-to (JP)

(72) Inventors: Hideaki IMAMURA, Tokyo-to (JP); Takuya SHIBAYAMA, Tokyo-to (JP)

(73) Assignee: Preferred Networks, Inc., Tokyo-to (JP)

(21) Appl. No.: 19/417,805

(22) Filed: Dec. 12, 2025

Related U.S. Application Data
(63) Continuation of application No. PCT/JP2024/021332, filed on Jun. 12, 2024.

(30) Foreign Application Priority Data
Jun. 13, 2023 (JP) 2023-096910

(51) Int. Cl. G16C 20/40; G16C 10/00; G16C 20/70

(52) U.S. Cl. CPC G16C 20/40; G16C 10/00; G16C 20/70

(57) ABSTRACT

An information processing device includes at least one memory and at least one processor. The at least one processor is configured to acquire information about a search target including at least information on a chemical element from another information processing device, repeatedly execute acquisition of a plurality of structures of a substance each containing the chemical element, update of the plurality of structures of the substance, and evaluation of the updated plurality of structures of the substance using a neural network, until a predetermined condition is satisfied, and after the repeated execution, transmit information about a searched structure of a substance and information about an evaluation value of the searched structure of the substance to the other information processing device. The at least one processor executes the acquisition based on the evaluation in the repeated execution.

### CROSS REFERENCE TO THE RELATED APPLICATION

This application is continuation application of International Application No. JP2024/021332, filed on Jun. 12, 2024, which claims priority to Japanese Application No. 2023-096910, filed on Jun. 13, 2023, the entire contents of which are incorporated herein by reference.

### FIELD

The present disclosure relates to an information processing device, an information processing method, and a non-transitory computer readable medium.

### BACKGROUND

It is desired, in a wide range of fields, to know a structure such as an arrangement and a bonding state or a physical property value of atoms constituting a crystal or the like. Today, a method of searching for a structure for a compound is actively researched and is a field where higher speed and higher accuracy are desired.

### BRIEF DESCRIPTION OF DRAWINGS

FIG. 1 shows a block diagram schematically illustrating an information processing device according to an embodiment.

FIG. 2 shows a block diagram schematically illustrating an information processing system according to an embodiment.

FIG. 3 shows a block diagram schematically illustrating an information processing system according to an embodiment.

FIG. 4 shows a diagram schematically illustrating a flow of a process according to an embodiment.

FIG. 5 shows a chart schematically illustrating a flow of sampling according to an embodiment.

FIG. 6 shows a chart schematically illustrating a flow of processes according to an embodiment.

FIG. 7 shows a diagram illustrating at least one implementation of the information processing device according to an embodiment.

### DETAILED DESCRIPTION

According to one embodiment, an information processing device includes at least one memory and at least one processor. The at least one processor is configured to acquire information about a search target including at least information on a chemical element from another information processing device, repeatedly execute acquisition of a plurality of structures of a substance each containing the chemical element, update of the plurality of structures of the substance, and evaluation of the updated plurality of structures of the substance using a neural network, until a predetermined condition is satisfied, and after the repeated execution, transmit information about a searched structure of a substance and information about an evaluation value of the searched structure of the substance to the other information processing device. And the at least one processor executes the acquisition based on the evaluation in the repeated execution.

A problem to be solved by embodiments of the present disclosure is not limited to the above-described problem and can also be a problem corresponding to effects described in the embodiments as some non-limiting examples of the problem. In other words, the problem corresponding to at least arbitrary one of the effects described in the explanation of the embodiments in the present disclosure can be the problem to be solved in the present disclosure.

Hereinafter, embodiments of the present invention will be explained with reference to the drawings. The drawings and the description of the embodiments are indicated as examples and are not intended to limit the present invention.

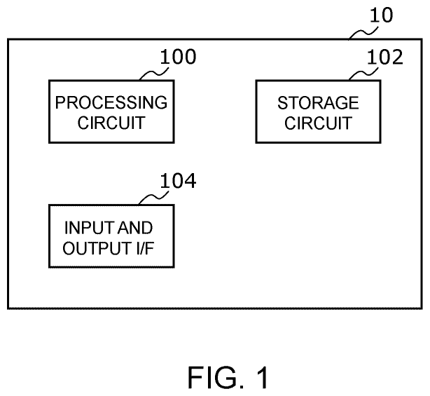

FIG. 1 is a block diagram schematically illustrating an information processing device according to an embodiment. An information processing device 10 includes a processing circuit 100, a storage circuit 102, and an input and output I/F 104. The information processing device 10 searches for the structure of a compound based on input information and outputs it.

The processing circuit 100 is a circuit (processor) which realizes processes in the information processing device 10. The processing circuit 100 executes various processes based on input information and/or data stored in the storage circuit 102.

The storage circuit 102 stores data required for the processes by the processing circuit 100 and/or data required for activating the information processing device 10.

The input and output I/F 104 is an interface which connects the outside and the inside of the information processing device 10. As a part of the input and output I/F 104, a user interface may be provided.

In addition to the above, the information processing device 10 may include configurations required for the information processing device 10 to execute the processes, such as a power supply unit and a control unit as appropriate.

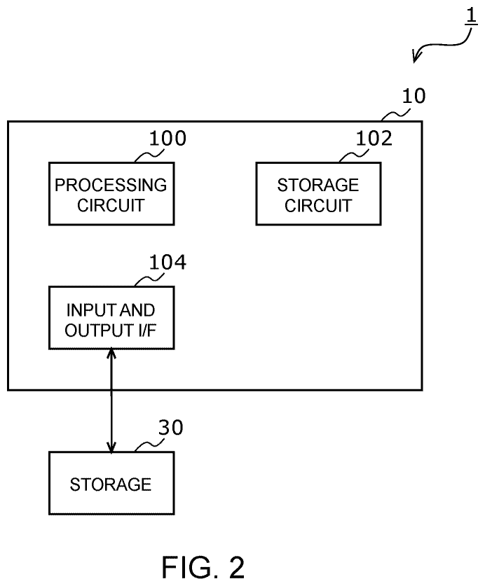

FIG. 2 is a diagram schematically illustrating an example of an information processing system according to an embodiment. The information processing system 1 includes the information processing device 10 and a storage 30.

The storage 30 can store at least part of the data required for the processes by the processing circuit 100. The processing circuit 100 can acquire the data stored in the storage 30 via the input and output I/F 104 and execute processes. The storage 30 may be configured to store data, such as a file server or a part of a server provided on a cloud.

As illustrated in this drawing, not the storage circuit 102 stores all the data required for the processes by the processing circuit 100 inside the information processing device 10, but the storage 30 provided outside the information processing device 10 can store the data processed by the processing circuit 100 and/or the intermediately acquired data and so on in the processes by the processing circuit 100. As explained above, the information processing system 1 can be configured such that a part of the function of the storage circuit 102 exists outside the information processing device 10.

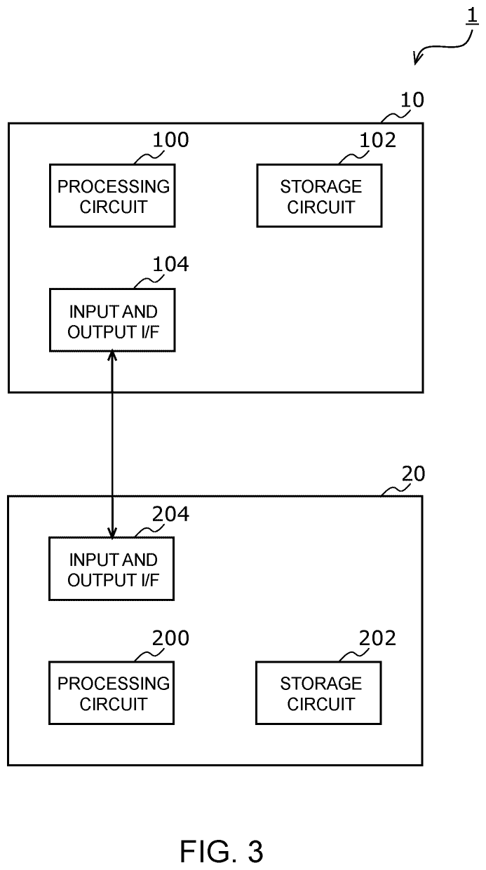

FIG. 3 is a diagram schematically illustrating an example of an information processing system according to an embodiment. An information processing system 1 includes at least an information processing device 10 and an information processing device 20.

The information processing device 10 is an information processing device which operates, for example, as a server, and the information processing device 20 is an information processing device which operates, for example, as a client.

The information processing device 20 includes a processing circuit 200, a storage circuit 202, and an input and output I/F 204. The information processing device 20 transmits information to the information processing device 10 and requests the information processing device 10 to search for a structure of a compound. The information processing device 10 executes a search for the structure of the compound based on the information and request acquired from the information processing device 20.

The processing circuit 200 is a circuit (processor) which executes processes in the information processing device 20. The processing circuit 200 accepts, for example, the request from a user via the input and output I/F 204, and transmits the request to the information processing device 10 via the input and output I/F 204.

The storage circuit 202 stores data necessary for the operation of the information processing device 20. Further, the storage circuit 202 can also store an operation result of the information processing device 10 acquired via the input and output I/F 204.

The input and output I/F 204 is an interface which connects the outside and the inside of the information processing device 20.

The input and output I/F 104 and the input and output I/F 204 can be connected, for example, via a wired or wireless network. In this case, the input and output I/F 104 and the input and output I/F 204 include an appropriate network I/F.

The information processing system 1 may additionally include the storage 30 in FIG. 2 and may include one or a plurality of information processing devices 10 for one or a plurality of information processing devices 20. In the case where the plurality of information processing devices 10 are included, a plurality of processors may process, in cooperation, the requests from the one or plurality of information processing devices 20.

In the case of this embodiment, the information processing device 10 may be an information processing device provided on the cloud and may be a device which provides a service in the form of Saas (Software as a Service) to the information processing device 20.

A stable structure has energy lower than that of a structure in the neighborhood. A compound having a structure lower in energy when a plurality of chemical elements are mixed can be said to be a stable (crystal) structure. By referring to a graph representing its state, what composition rate constitutes a stable structure can be known. For example, a structure on a convex hull can be said to be a stable structure.

In the present disclosure, the search for the structure of a compound by updating the convex hull is a non-limiting object. One of the non-limiting problems to be solved by the embodiment of the present disclosure is to search for a structure of a substance having a structure more stable in terms of energy or a structure including a desired physical property value, as the structure search.

In the present disclosure, the substance is various types of materials or the like and is a concept including a compound or a simple substance. For example, the substance may be a crystal of a simple substance, a carbon nanotube, fullerene, nanoparticle, or the like. As the structure search of the substance, for example, a structure of a surface of a substance, a molecular structure, or the like, may be searched for. In the case of simply describing a “structure” in the following, it may be read as an atomic structure including an atomic arrangement, an atomic array, or the like of this substance.

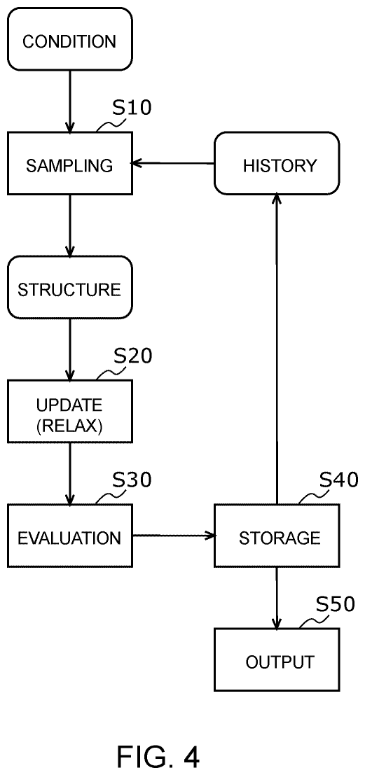

FIG. 4 is a diagram schematically illustrating a flow of a substance structure search process by the information processing device 10. In the following, this may be described as the process by the information processing device 10 or the information processing device 20, and can be read as the process by the processing circuit 100 or the processing circuit 200 in a consistent range.

The information processing device 10 samples a structure based on a given condition (S10).

The information processing device 10 updates (relaxes) the sampled structure (20).

The information processing device 10 evaluates the updated structure (S30).

The information processing device 10 stores the structure as a history with an evaluation (S40).

The information processing device 10 performs sampling based on the evaluation from the history in iteration (S10 to S40) in or after a second round. Further, the information processing device 10 outputs a structure according to an evaluation value when a predetermined operation completion condition is satisfied (S50).

Each process in the above processes will be explained in more detail.

### (Initialization of the History)

The information processing device 10, when executed the search by a combination of the same chemical elements in the past, may use the result in the search in the past or the information about an interim progress, as the history.

In the case of executing a new search according to a combination of chemical elements which has not been executed before or executing a new search not according to the past result on a combination which has been executed before, the information processing device 10 can regard a structure, in which chemical elements are randomly arranged, as an initial structure. For example, the information processing device 10 can use, as an initial history, a plurality of structures in which chemical elements acquired as a condition are randomly arranged in a cell in a DFT (Density Functional Theory) calculation.

The information processing device 10 can arrange chemical elements by dividing a cell into a plurality of sub-cells, randomly arranging chemical elements in one sub-cell, and copying the sub-cells and filling the cell with them, as a non-limiting example.

### (S10: Sampling of the Structure)

The information processing device 10 can use an evolutionary algorithm as a non-limiting example in the sampling of the structure. The information processing device 10 samples the structure using, for example, a genetic algorithm.

The genetic algorithm, which is one of the evolutionary algorithms, selects samples from a plurality of samples, generally a vast number of parent generation candidates, and forms a parent generation. In the samples belonging to the parent generation, crossover of the samples and mutations in the samples are performed to create candidates of samples for a child generation.

The wording of sampling of the structure or simple sampling in the present disclosure indicates the process of sample creation for the child generation via the selection of the sample in the parent generation and the process including at least the crossover (mutation may be made).

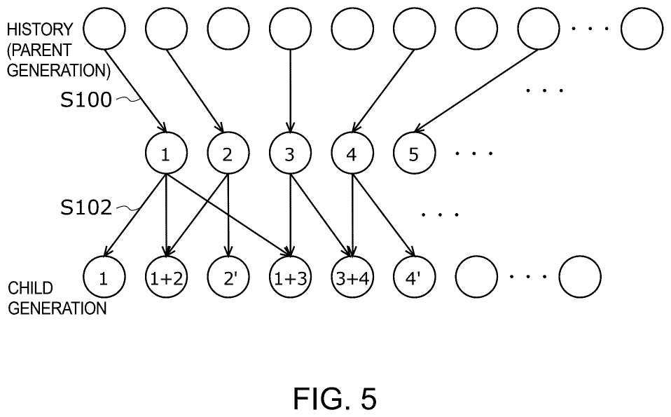

FIG. 5 is a chart illustrating an example of the sampling according to an embodiment. The information processing device 10 creates the child generation from history information and executes a subsequent process with the child generation as the sampled structure.

The information processing device 10 prepares the child generation creation by elite selection with the history as the parent generation (S100). The elite selection is executed based on the evaluation associated with the sample in the history.

The information processing device 10 can perform selection using, for example, a value of energy for each structure (sample) acquired as the evaluation in the previous iteration. For example, in the case where a crystal structure composed of two chemical elements is desired to be acquired, a two-dimensional composition on the convex hull can be selected as an elite sample with the ratio of the two chemical elements as a horizontal axis and the energy [eV/atom] in the structure in each of the various arrangements at the chemical element ratio as a vertical axis.

In addition, the information processing device 10 can also perform selection based on a method of multi-objective optimization using not only energy but also an index regarding the stability such as a plurality of physical property values or a desired physical property. In this case, the information processing device 10 can also perform the selection using other evaluation values acquired in the process of evaluation at S30 explained later as a non-limiting example.

The information processing device 10 can perform the elite selection based on a partially ordered sequence using a predetermined index as a sort key, as a non-limiting example. A partial order is generally a relation where a reflectance, an anti-symmetric law, and a transitive law are established.

In one embodiment, p>=q with respect to structures p and q is assumed to mean that when E(p)<=E(q) and Comp(p)=Comp(q), <=satisfies a partial order law. Here, E(p) indicates energy of the structure p or a value of an index of sort, and Comp(p) indicates a composition rate of p (for example, a composition ratio of the two chemical elements). Specifically, in other words, the case where the value of the index indicates E(p)<=E(q) with respect to different samples p and q having the same composition rate is described as p<=q.

The information processing device 10 calculates a value E being an index for each of the structures of the samples stored in the history. The information processing device 10 executes non-dominated sorting for E, to extract samples in ascending order of rank. For example, when there are a plurality of structures having the same chemical element composition rate, the information processing device 10 sets ranks for the structures in ascending order of index and selects the structure as a sample in ascending order of rank.

The information processing device 10 can further perform selection in ascending order of rank, and perform selection of a sample using a crowding distance D at a rank exceeding the number of samples to be selected. The crowding distance D of the sample p can be, for example, D(p)=(Manhattan distance from two samples in a p neighborhood), D(p)=(average of Manhattan distances from a plurality of samples in p neighborhood), or D(p)=(Manhattan distances from a plurality of samples in p neighborhood)+ (normalized energy gap of p), or the like. The neighborhood indicates other samples close in index, for example, the value of energy.

This selection allows the information processing device 10 to execute the elite selection, and the thus-selected sample is always an endpoint of the convex hull in a multidimensional space (for example, a space formed with an energy value with respect to the composition rate of a plurality of chemical elements). Therefore, the selected sample is more likely to be a sample showing a structure that has not improved during a long iteration.

The information processing device 10 can perform filtering on the sample before the non-dominated sorting in order to appropriately select or not to select the sample. The filtering can be expressed, for example, by the following expressions.

$$\begin{matrix}[ Math 1 ] & \\ normalized ( E ( t ) ) \times {α}^{g ( t ) - {g}^{*} ( t )} \leq ε & ( 1 )\end{matrix}$$

$$\begin{matrix}[ Math 2 ] & \\ normalized ( E ( t ) ) = \frac{{E}_{max} - E ( t )}{{E}_{max} - {E}_{min}} & ( 2 )\end{matrix}$$

g(t) is a current generation of t, and g*(t) is a generation of t satisfying the following.

$$\begin{matrix}[ Math 3 ] & \\ arg min \{ E ( s ) \vert s ∈ history , \vert r ( t ) - r ( s ) \vert \leq δ \} & ( 3 )\end{matrix}$$

history represents the whole structure (history) searched for till then. r(t) is a multidimensional vector representing a composition of a structure t, and α, ε, and δ are hyperparameters. Further, |⋅| indicates a Manhattan norm of the multidimensional vector. The information processing device 10 removes, for example, t satisfying Expression (1) from the candidates of the elite selection.

By executing the non-dominated sorting and the crowding distance sorting for the thus-selected candidates, the information processing device 10 can execute the elite selection. By this selection, it becomes possible to remove individuals belonging to a region where many structures themselves exist in the neighborhood, namely, many individuals having similar genes have been already evaluated, from the candidates, and preferentially select the individuals in a region, where the search has not been executed so much in the history, which is the other region, as samples for the next generation.

After the selection of the samples, the information processing device 10 creates the samples for the child generation by the selected samples themselves, crossover of a plurality of selected samples, and mutations in the selected samples (S102).

In the case of using the selected samples themselves, the information processing device 10 uses the structure in the history of the parent generation as it is as one sample of the structure of the child generation. For example, Sample 1 selected as the elite in the drawing is regarded as the structure belonging to the child generation.

When crossing over the samples, the information processing device 10 replaces at least a selected part of the structure of the parent generation with another selected part of the structure of the parent generation. For example, a part of the structure of Sample 1 in the drawing is replaced with a part of the structure of Sample 3 into a sample indicated with 1+3 in the child generation.

Crossover can be executed in units of cells, for example, in the DFT calculation or the like. In a structure indicated by a cell in a sample, by replacing the structure of a chemical element in at least a part of a region thereof with the structure of the chemical element in a part of a region of another sample, a crossed-over sample can be acquired.

Further, the crossover is not limited to the above, but may be executed, for example, by exchanging a part of the structure represented by a graph with a part of the structure of another graph, or exchanging a part of a structure described in SMILES with another part of the structure described in SMILES.

When mutating the sample, the information processing device 10 changes the type of at least part of chemical elements in the structure in a sample, the arrangement of at least part of chemical elements, or the connection among at least part of chemical elements, to another one. For example, the structure of Sample 4 in the drawing is mutated into a sample indicated with 4′.

The information processing device 10 can replace two different types of chemical elements in the structure, for example, in one sample, to cause a mutation. The information processing device 10 can cause a mutation, for example, by moving the positions of four chemical elements having a rectangular shape in one sample in a manner to form a parallelogram. As other examples, the information processing device 10 can arrange (add) or delete random chemical elements at random positions, or randomly change the types of chemical elements to cause a mutation.

In addition, though not illustrated, both the crossover and the mutation may be executed for two samples to create one sample in the child generation. For example, Sample (1+2)′ in the child generation may be created from Sample 1 and Sample 2.

As explained above, the information processing device 10 executes the sampling of the structure of the substance by the genetic algorithm. As a matter of course, the information processing device 10 can execute the sampling similarly by a method of another evolutionary algorithm.

Further, the information processing device 10 can execute the sampling also by a method based on a surrogate model such as Bayesian optimization, a sampling-based method such as a Markov Chain Monte Carlo method (MCMC), or a method based on a symmetry of space groups.

### (S20: Update (Relaxation), S30: Evaluation)

After the sampling, the information processing device 10 updates the sample so that the structure is established as a stable structure or a structure having a desired physical property value for each of the sampled structures.

The structure subjected to the above crossover and mutation may not be stable as a crystal structure, for example, such that the distance between atoms is too short. To update the created unstable structure to a stable structure, the information processing device 10 executes a relaxation process.

The information processing device 10 can locally optimize the sampled structure, for example, using NNP (Neural Network Potential).

The information processing device 10 applies NNP for the sampled structure to calculate energy. The information processing device 10 executes the optimization of the structure to make it a locally stable structure so that its energy value becomes small. The information processing device 10 can execute the optimization of the local structure, for example, by finding a position differentiation with respect to energy.

By this process, the information processing device 10 updates (relaxes) the sample acquired by the crossover and/or the mutation with a stable structure in the neighborhood of the sample. In other words, the information processing device 10 updates (relaxes) the sample to a more stable structure.

More specifically, the information processing device 10 updates the sample to a sample indicating a point where the gradient of the potential of NNP becomes 0, by applying a BFGS method (Broyden-Fletcher-Goldfarb-Shanno Algorithm) or the like, using the target sample as an initial value. The neighborhood may indicate some region including the point where the gradient becomes 0 and the sample (before update). What shape the region has differs depending on the shape of a potential curved surface of NNP and the position of the sample.

The information processing device 10 can employ any appropriate method as the optimization method. The information processing device 10 can acquire the stable structure from the energy value of the structure in the neighborhood, for example, by locally employing the BFGS method. In addition, the information processing device 10 relaxes the structure using a hill climbing method, a quasi-Newton method, or the like to update the sampled structure.

This process makes it possible to update the structure that is likely to be inappropriate in the structure acquired from the genetic algorithm to a more stable structure and execute a search.

Note that the information processing device 10 can also evaluate the sampled structure at S30 in the process at S20. Specifically, the evaluation value acquired for the optimization in the process at S20 can be regarded as an evaluation value at S30.

In the case of using an index different from the evaluation used in the local optimization at S20, as the evaluation at the process at S10, a calculation process of the index can be performed on the sample updated after completion of the process at S20. Also in this case, if the evaluation value used in the process at S20 is a part of the evaluation at S30, the evaluation value acquired during the interim progress at S20 can be used.

The information processing device 10 can evaluate the sampled structure, for example, by calculating the energy of each of relaxed structures and finding the difference between the calculated energy and the energy of a true convex hull.

The search is generally executed in a situation in which the true convex hull is unknown, and therefore the information processing device 10 can calculate the distance from the convex hull with the current energy to the calculated energy of each of the structures, for example, the distance to the structure belonging to the current convex hull to perform the evaluation.

Further, as another example, the information processing device 10 can also regard the energy value that is known, for example, published as a database as the convex hull, and use the distance between the convex hull and the convex hull in the sampled structure, as the evaluation.

Further, as another example, the information processing device 10 can use the area or the line length of the convex hull as the whole evaluation, or can use the distance between a point indicating a configuration and the convex hull as the evaluation.

### (S40: Store, S50: Output)

The information processing device 10 stores the evaluated result and the evaluated structure in association (S40). The stored structure is registered as a history and can be used as a sample in the parent generation in the next iteration.

The information processing device 10 repeats the processes from S10 to S40 until an end condition is reached. The end condition can be set similarly to the general optimization such that the number of structures that have been properly evaluated reaches a predetermined number, the time required for the search has passed a predetermined time, a predetermined physical property value of the searched structure reaches a predetermined value, or a predetermined number of times of iteration is executed. After the end condition is satisfied, the information processing device 10 outputs information on the finally acquired sample (S50).

The information processing device 10 can output, for example, a structure for the sample constituting the convex hull, and can also output a structure in which the distance from the convex hull is within a predetermined value, for example, within a value of 0.05 [eV/atom]. In this configuration, an error occurs in operations of NNP, DFT, and the like, and the configuration within the above predetermined value can be actually synthesized, and, in such a case, output of the candidate of the search result generates meaning. Besides, in the case of performing a search based on the physical property value, it is conceivable to return the stable or metastable structure in which the physical property value falls within the desired range.

By optimization and output relating to the convex hull which can be acquired for the evaluation or sampling, the information processing device 10 can execute a search based on a Pareto front in other variable optimization or a search based on the optimization of a pseudo-Pareto front, and output its result.

The information processing device 10 can output the type of a chemical element, positions of atoms, and a cell structure which are parameters deciding the crystal structure, as the information about the structure. Further, the information processing device 10 can output the energy or the physical property value.

The information processing device 10 may further output the history of the search. In this case, it is possible to present to the user the information about how the search has been executed, for example, whether an efficient search has been executed.

Further, the information processing device 10 can output information for presenting a viewer to visualize the structure. This output can be presented, for example, via the input and output I/Fs 104, 204.

Note that the information processing device 10 can also output the interim progress as appropriate also during the execution of the search. The information processing device 10 can also output the information not only on the structure but also on the evaluation value as a graph or output the state of the convex hull as a graph.

According to this embodiment, the information processing device 10 can automatically search for the structure of the substance composed of the chemical elements, for example, only by inputting conditions such as the types of chemical elements to be used. Therefore, the information processing system 1 illustrated in FIG. 3 can execute a high-speed operation on a server side only by inputting the information on the types of chemical elements (for example, a substance containing Li and In, a crystal containing It and O) from the client side, and provide a service as the SaaS of outputting the search result to a client.

The operation based on a classical theory of acquiring a value which can be the evaluation value of the energy or the like, such as the first-principles calculation, a calculation by molecular dynamics, or a calculation by the density functional theory requires a long time in the operation relating to each structure.

In the case of using the genetic algorithm as a non-limiting example as the sampling, the information processing device 10 may execute a plurality of times of the operation for a very long time in order to evaluate a plurality of samples in the child generation, and may require a time such as several days to several weeks, for example, in order to acquire the evaluation values for one generation to acquire a stable structure.

By using the method of NNP, the calculation of the evaluation value and the relaxation process of the structure can be speedily and accurately realized. As a result of this, according to one embodiment, a global and robust search for the structure of a substance using a method of the evolutionary algorithm such as a high-speed genetic algorithm can be realized.

In the case of using the genetic algorithm as an example of the sampling, the optimization can be realized at the same time for a plurality of samples. As a result of being able to increase the number of samples that can be sampled, the high-speed and global structure search can be realized. The use of NNP makes it possible to speedily realize the evaluation that has been a bottleneck in the search using the plurality of samples at the same time. In one embodiment of the present disclosure, the use of NNP as above makes it possible to speedily realize the evaluation, while ensuring the robustness of the structure search for the substance.

The use of the plurality of samples at the same time makes it possible to realize a more accurate process. Further, the increase in speed of the evaluation by NNP makes it possible to increase the scope of the search more than before, by a plurality of accelerators such as general GPUs, without using a supercomputer.

Besides, the information which can be input as the condition is not the combination of chemical elements. For example, the information processing device 10 may accept the information about the environments such as temperature and pressure as the conditions. In this case, the information processing device 10 can reflect the information about the environments such as temperature and pressure in the acquisition of the physical property value in NNP, and can accurately realize the local search for the stable structure under the conditions. As a result of this, the information processing device 10 can execute the search result as the whole process as much as possible under the conditions.

The information input as the conditions can be input by, for example, the user via the information processing device 20 on the client side.

As the conditions, the combination of chemical elements and the composition ratio of the chemical elements may be set. The composition ratio may be expressed by a range. In this case, the information processing device 10 can execute the process based on the composition ratio (for example, the initial sample creation, crossover, mutation) at the timing of sampling as a non-limiting example. When the number of chemical elements is large, the search range can be limited.

The information processing device 10 can perform sampling, for example, with the range of the composition ratio of Li to In decided. This sampling allows a search for the structure of the substance in different states such as a charging state and a discharging state.

The information processing device 10 can perform sampling, for example, with the range of the presence ratio of chemical elements between a plurality of layers constituting a semiconductor. This sampling allows a search for a fixed structure such as an atomic arrangement between the layers of the semiconductor. The semiconductor may change in physical property value even if the amount of atoms to be converted is minute, but the search can be executed while covering the change in the physical property value.

The information processing device 10 can perform sampling, with the range in the curve formed by the already acquired convex hulls designated as an example of the search range. This sampling allows the search in the designated range with high granularity.

As the condition, the ranges of the physical property values such as energy and density may be set. In this case, the information processing device 10 can execute the local optimization in the case of using NNP with higher accuracy. Further, in the evaluation using the multi-objective optimization, a structure closer to the condition can be acquired.

As the condition, the possibility of synthesis for the acquired structure can be output. The possibility of the synthesis may be the one determined based on an existing process.

As the condition, the evaluation function to be used may be settable.

The information processing device 10 can output a new structure itself as explained above. In addition, the information processing device 10 can also output at least one of the energy to be used for evaluation or the state of the convex hull of another variable, a visualization from which parent each structure has been acquired (including a change over time), the history of optimization, and the physical property value of the structure (crystal group, cell structure).

According to the above embodiment, the calculation of the physical property value for evaluation can be speedily realized using NNP, and therefore the number of individuals in one generation of the evolutionary algorithm can be increased. In other words, it is possible to further increase the degree of parallelism in the case of using the accelerator using a plurality of cores, using the cluster, and using the grid computing. As a result of this, it becomes possible to improve the operation speed and improve the accuracy with a wider search range.

In the case of using the configuration in FIG. 3, the information processing device 10 may execute the search by the evolutionary algorithm, and the information processing device 20 may execute the evaluation. Further, as another example, the information processing device 10 may execute the search by the evolutionary algorithm, and another information processing device 10 may execute the evaluation. As explained above, each process can be executed in an appropriate information processing device as needed.

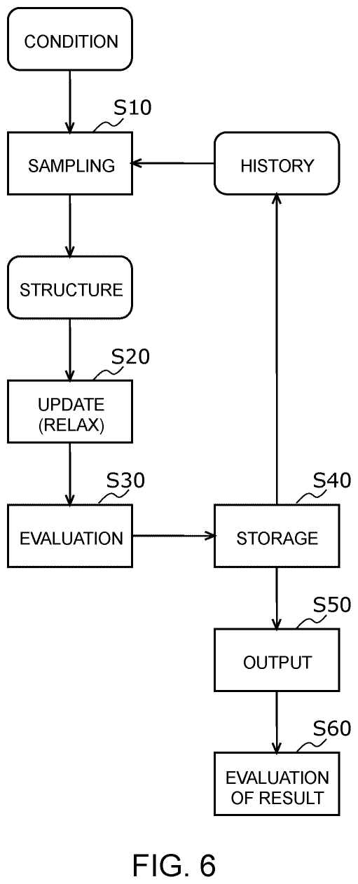

FIG. 6 is a chart illustrating an embodiment for evaluating the result output in the above embodiment. The information processing device 10 in FIG. 1 to FIG. 3 or the information processing device 20 in FIG. 3 can evaluate the result of completion of the search (S60).

The information processing device 10 or the information processing device 20 may use, for example, the structure of the sample output as the search result as a candidate of the structure desired to be acquired, in which case a most desirable structure can be extracted from among the candidates by an evaluation on a plurality of candidate structures acquired in the search.

For example, in addition to the evaluation at S30, the physical property value such as energy can be found by a classical DFT operation. By the evaluation, the energy value or the like is calculated using another operation method for the candidate searched for at high speed using NNP, thereby making it possible to increase the accuracy that the desired structure has been acquired.

Further, in the case using the known convex hull as the target of evaluation or the like, the information processing device 10 or the information processing device 20 may calculate the value of the energy or the like for the structure belonging to the known convex hull by a classical method or the method using NNP. By acquiring the energy value or the like of the existing structure as above, it becomes possible to confirm that the evaluation in the information processing device 10 has been appropriately performed.

The information processing device 10 acquires at least the information about the search target from the information processing device 20.

The information about the search target presents the information acquired from the information processing device 20 used by the user. The information about the search target can include information such as the types and the composition ratio of chemical elements constituting the substance which are described in the embodiment. The information is not limited to them but can also include some information indicating the substance being the search target and some information indicating search conditions (the temperature, pressure, the range of the convex hull, the range of the physical property value, and so on). Further, the information processing device 10 may acquire the information about the search target from the information processing device 20 via another device.

The information processing device 10 acquires a plurality of structures of a substance based on the information about the acquired search target and updates the acquired plurality of structures of the substance.

The acquisition of the plurality of structures of the substance may be a concept including at least one of the following.

Specifically, the information processing device 10 may acquire the plurality of structures of the substance by sampling.

Besides, the information processing device 10 may acquire the plurality of structures of the substance by a random arrangement.

Besides, the information processing device 10 may acquire the plurality of structures of the substance based on the history information. Also in the case of performing the elite selection using the history, the information processing device 10 may make at least one of the candidates to be selected the structure of the substance created by the random arrangement. This can incorporate a global search into a local search.

Further, the information on the structure of the substance may be the concept including at least one of the following.

Specifically, the information on the structure of the substance may be information including the type of the atom (chemical element) and the positional information on the atom (chemical element).

Besides, the information on the structure of the substance may be information including information on the cell structure.

Note that the information processing device 10 may be configured to acquire at least one structure of the substance based on the acquired information about the search target and update the at least one acquired structure of the substance.

The information processing device 10 can evaluate the updated plurality of structures of the substance using the neural network (for example, NNP) and transmit the information on the searched structure of the substance to the information processing device 20 based on the evaluation result. Further, the information processing device 10 may transmit the information on the structure of the substance searched for by the information processing device 20, via another device.

NNP may be considered as one type of the neural network.

It is not excluded that NNP is constituted using a neural network model as well as a neural network constituting a graph based on a so-called atomic arrangement and the structure is evaluated by the other neural network model. In other words, the NNP in the present disclosure does not exclude the use of the neural network model for acquiring the information on the structure of the substance as a wider concept.

The information processing device 10 can update the acquired plurality of structures of the substance using the neural network.

This neural network may be a neural network of the above NNP or may be a model different therefrom.

Note that the information processing device 10 may be configured to evaluate the information on the updated at least one structure of the substance, using the neural network.

Besides, the information processing device 10 may be configured to transmits the information on the searched one or plurality of structures of the substance to the information processing device 20.

A plurality of information processing devices 20 may be provided as explained also in the above.

Similarly, the information processing device 10 may be configured as a system including a plurality of devices.

The information processing system 1 explained in the present disclosure may include one or a plurality of information processing devices 10 and one or a plurality of information processing devices 20. Similarly, the information processing system 1 can further include one or a plurality of storages 30.

In the information processing system 1, these configurations are connected by a network line using a wireless or wired means including typically the Internet, but the connection method is not limited to them and a means capable of properly connecting them can be used.

As an example of the configuration, the information processing system 1 can also provide a SaaS using the information processing device 10 as a server and the information processing device 20 as a client.

Some or all of each device (the information processing device 10 or the information processing device 20) in the above embodiment may be configured in hardware, or information processing of software (program) executed by, for example, a CPU (Central Processing Unit), GPU (Graphics Processing Unit). In the case of the information processing of software, software that enables at least some of the functions of each device in the above embodiments may be stored in a non-volatile storage medium (non-volatile computer readable medium) such as CD-ROM (Compact Disc Read Only Memory) or USB (Universal Serial Bus) memory, and the information processing of software may be executed by loading the software into a computer. In addition, the software may also be downloaded through a communication network. Further, entire or a part of the software may be implemented in a circuit such as an ASIC (Application Specific Integrated Circuit) or FPGA (Field Programmable Gate Array), wherein the information processing of the software may be executed by hardware.

A storage medium to store the software may be a removable storage media such as an optical disk, or a fixed type storage medium such as a hard disk, or a memory. The storage medium may be provided inside the computer (a main storage device or an auxiliary storage device) or outside the computer.

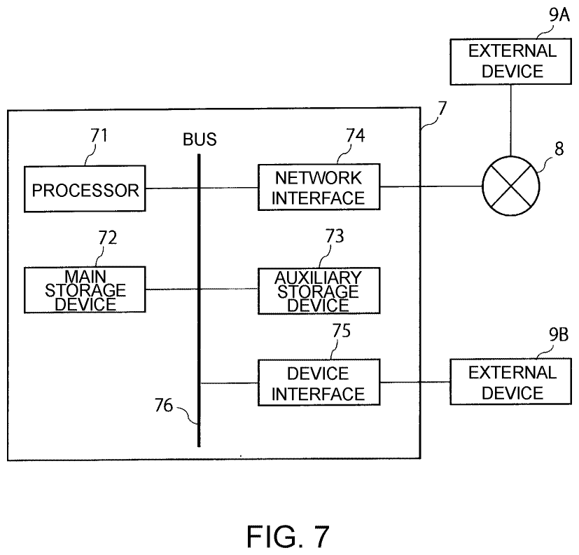

FIG. 7 is a block diagram illustrating an example of a hardware configuration of each device (the information processing device 10 or the information processing device 20) in the above embodiments. As an example, each device may be implemented as a computer 7 provided with a processor 71, a main storage device 72, an auxiliary storage device 73, a network interface 74, and a device interface 75, which are connected via a bus 76.

The computer 7 of FIG. 7 is provided with each component one by one but may be provided with a plurality of the same components. Although one computer 7 is illustrated in FIG. 7, the software may be installed on a plurality of computers, and each of the plurality of computer may execute the same or a different part of the software processing. In this case, it may be in a form of distributed computing where each of the computers communicates with each of the computers through, for example, the network interface 74 to execute the processing. That is, each device (the information processing device 10 or the information processing device 20) in the above embodiments may be configured as a system where one or more computers execute the instructions stored in one or more storages to enable functions. Each device may be configured such that the information transmitted from a terminal is processed by one or more computers provided on a cloud and results of the processing are transmitted to the terminal.

Various arithmetic operations of each device (the information processing device 10 or the information processing device 20) in the above embodiments may be executed in parallel processing using one or more processors or using a plurality of computers over a network. The various arithmetic operations may be allocated to a plurality of arithmetic cores in the processor and executed in parallel processing. Some or all the processes, means, or the like of the present disclosure may be implemented by at least one of the processors or the storage devices provided on a cloud that can communicate with the computer 7 via a network. Thus, each device in the above embodiments may be in a form of parallel computing by one or more computers.

The processor 71 may be an electronic circuit (such as, for example, a processor, processing circuitry, processing circuitry, CPU, GPU, FPGA, or ASIC) that executes at least controlling the computer or arithmetic calculations. The processor 71 may also be, for example, a general-purpose processing circuit, a dedicated processing circuit designed to perform specific operations, or a semiconductor device which includes both the general-purpose processing circuit and the dedicated processing circuit. Further, the processor 71 may also include, for example, an optical circuit or an arithmetic function based on quantum computing.

The processor 71 may execute an arithmetic processing based on data and/or a software input from, for example, each device of the internal configuration of the computer 7, and may output an arithmetic result and a control signal, for example, to each device. The processor 71 may control each component of the computer 7 by executing, for example, an OS (Operating System), or an application of the computer 7.

Each device (the information processing device 10 or the information processing device 20) in the above embodiments may be enabled by one or more processors 71. The processor 71 may refer to one or more electronic circuits located on one chip, or one or more electronic circuitries arranged on two or more chips or devices. In the case of a plurality of electronic circuitries is used, each electronic circuit may communicate by wired or wireless.

The main storage device 72 may store, for example, instructions to be executed by the processor 71 or various data, and the information stored in the main storage device 72 may be read out by the processor 71. The auxiliary storage device 73 is a storage device other than the main storage device 72. These storage devices shall mean any electronic component capable of storing electronic information and may be a semiconductor memory. The semiconductor memory may be either a volatile or non-volatile memory. The storage device for storing various data or the like in each device (the information processing device 10 or the information processing device 20) in the above embodiments may be enabled by the main storage device 72 or the auxiliary storage device 73 or may be implemented by a built-in memory built into the processor 71. For example, the storage circuit 102 or the storage circuit 202 in the above embodiments may be implemented in the main storage device 72 or the auxiliary storage device 73.

In the case of each device (the information processing device 10 or the information processing device 20) in the above embodiments is configured by at least one storage device (memory) and at least one processor connected/coupled to/with this at least one storage device, the at least processor may be connected to a single storage device. Or the at least storage may be connected to a single processor. Or each device may include a configuration where at least one of the plurality of processors is connected to at least one of the plurality of storage devices. Further, this configuration may be implemented by a storage device and a processor included in a plurality of computers. Moreover, each device may include a configuration where a storage device is integrated with a processor (for example, a cache memory including an L1 cache or an L2 cache).

The network interface 74 is an interface for connecting to a communication network 8 by wireless or wired. The network interface 74 may be an appropriate interface such as an interface compatible with existing communication standards. With the network interface 74, information may be exchanged with an external device 9A connected via the communication network 8. Note that the communication network 8 may be, for example, configured as WAN (Wide Area Network), LAN (Local Area Network), or PAN (Personal Area Network), or a combination of thereof, and may be such that information can be exchanged between the computer 7 and the external device 9A. The internet is an example of WAN, IEEE802.11 or Ethernet (registered trademark) is an example of LAN, and Bluetooth (registered trademark) or NFC (Near Field Communication) is an example of PAN.

The device interface 75 is an interface such as, for example, a USB that directly connects to the external device 9B.

The external device 9A is a device connected to the computer 7 via a network. The external device 9B is a device directly connected to the computer 7.

The external device 9A or the external device 9B may be, as an example, an input device. The input device is, for example, a device such as a camera, a microphone, a motion capture, at least one of various sensors, a keyboard, a mouse, or a touch panel, and gives the acquired information to the computer 7. Further, it may be a device including an input unit such as a personal computer, a tablet terminal, or a smartphone, which may have an input unit, a memory, and a processor.

The external device 9A or the external device 9B may be, as an example, an output device. The output device may be, for example, a display device such as, for example, an LCD (Liquid Crystal Display), or an organic EL (Electro Luminescence) panel, or a speaker which outputs audio. Moreover, it may be a device including an output unit such as, for example, a personal computer, a tablet terminal, or a smartphone, which may have an output unit, a memory, and a processor.

Further, the external device 9A or the external device 9B may be a storage device (memory). The external device 9A may be, for example, a network storage device, and the external device 9B may be, for example, an HDD storage.

Furthermore, the external device 9A or the external device 9B may be a device that has at least one function of the configuration element of each device (the information processing device 10 or the information processing device 20) in the above embodiments. That is, the computer 7 may transmit a part of or all of processing results to the external device 9A or the external device 9B, or receive a part of or all of processing results from the external device 9A or the external device 9B.

In the present specification (including the claims), the representation (including similar expressions) of “at least one of a, b, and c” or “at least one of a, b, or c” includes any combinations of a, b, c, a-b, a-c, b-c, and a-b-c. It also covers combinations with multiple instances of any element such as, for example, a-a, a-b-b, or a-a-b-b-c-c. It further covers, for example, adding another element d beyond a, b, and/or c, such that a-b-c-d.

In the present specification (including the claims), the expressions such as, for example, “data as input,” “using data,” “based on data,” “according to data,” or “in accordance with data” (including similar expressions) are used, unless otherwise specified, this includes cases where data itself is used, or the cases where data is processed in some ways (for example, noise added data, normalized data, feature quantities extracted from the data, or intermediate representation of the data) are used. When it is stated that some results can be obtained “by inputting data,” “by using data,” “based on data,” “according to data,” “in accordance with data” (including similar expressions), unless otherwise specified, this may include cases where the result is obtained based only on the data, and may also include cases where the result is obtained by being affected factors, conditions, and/or states, or the like by other data than the data. When it is stated that “output/outputting data” (including similar expressions), unless otherwise specified, this also includes cases where the data itself is used as output, or the cases where the data is processed in some ways (for example, the data added noise, the data normalized, feature quantity extracted from the data, or intermediate representation of the data) is used as the output.

In the present specification (including the claims), when the terms such as “connected (connection)” and “coupled (coupling)” are used, they are intended as non-limiting terms that include any of “direct connection/coupling,” “indirect connection/coupling,” “electrical connection/coupling,” “communicative connection/coupling,” “operative connection/coupling,” “physical connection/coupling,” or the like. The terms should be interpreted accordingly, depending on the context in which they are used, but any forms of connection/coupling that are not intentionally or naturally excluded should be construed as included in the terms and interpreted in a non-exclusive manner.

In the present specification (including the claims), when the expression such as “A configured to B,” this may include that a physically structure of A has a configuration that can execute operation B, as well as a permanent or a temporary setting/configuration of element A is configured/set to actually execute operation B. For example, when the element A is a general-purpose processor, the processor may have a hardware configuration capable of executing the operation B and may be configured to actually execute the operation B by setting the permanent or the temporary program (instructions). Moreover, when the element A is a dedicated processor, a dedicated arithmetic circuit, or the like, a circuit structure of the processor or the like may be implemented to actually execute the operation B, irrespective of whether or not control instructions and data are actually attached thereto.

In the present specification (including the claims), when a term referring to inclusion or possession (for example, “comprising/including,” “having,” or the like) is used, it is intended as an open-ended term, including the case of inclusion or possession an object other than the object indicated by the object of the term. If the object of these terms implying inclusion or possession is an expression that does not specify a quantity or suggests a singular number (an expression with a or an article), the expression should be construed as not being limited to a specific number.

In the present specification (including the claims), although when the expression such as “one or more,” “at least one,” or the like is used in some places, and the expression that does not specify a quantity or suggests a singular number (the expression with a or an article) is used elsewhere, it is not intended that this expression means “one.” In general, the expression that does not specify a quantity or suggests a singular number (the expression with a or an as article) should be interpreted as not necessarily limited to a specific number.

In the present specification, when it is stated that a particular configuration of an example results in a particular effect (advantage/result), unless there are some other reasons, it should be understood that the effect is also obtained for one or more other embodiments having the configuration. However, it should be understood that the presence or absence of such an effect generally depends on various factors, conditions, and/or states, etc., and that such an effect is not always achieved by the configuration. The effect is merely achieved by the configuration in the embodiments when various factors, conditions, and/or states, etc., are met, but the effect is not always obtained in the claimed invention that defines the configuration or a similar configuration.

In the present specification (including the claims), when the term such as “maximize/maximization” is used, this includes finding a global maximum value, finding an approximate value of the global maximum value, finding a local maximum value, and finding an approximate value of the local maximum value, should be interpreted as appropriate accordingly depending on the context in which the term is used. It also includes finding on the approximated value of these maximum values probabilistically or heuristically. Similarly, when the term such as “minimize/minimization” is used, this includes finding a global minimum value, finding an approximated value of the global minimum value, finding a local minimum value, and finding an approximated value of the local minimum value, and should be interpreted as appropriate accordingly depending on the context in which the term is used. It also includes finding the approximated value of these minimum values probabilistically or heuristically. Similarly, when the term such as “optimize/optimization” is used, this includes finding a global optimum value, finding an approximated value of the global optimum value, finding a local optimum value, and finding an approximated value of the local optimum value, and should be interpreted as appropriate accordingly depending on the context in which the term is used. It also includes finding the approximated value of these optimal values probabilistically or heuristically.

In the present specification (including claims), when a plurality of hardware performs a predetermined process, the respective hardware may cooperate to perform the predetermined process, or some hardware may perform all the predetermined process. Further, a part of the hardware may perform a part of the predetermined process, and the other hardware may perform the rest of the predetermined process. In the present specification (including claims), when an expression (including similar expressions) such as “one or more hardware perform a first process and the one or more hardware perform a second process,” or the like, is used, the hardware that perform the first process and the hardware that perform the second process may be the same hardware, or may be the different hardware. That is: the hardware that perform the first process and the hardware that perform the second process may be included in the one or more hardware. Note that, the hardware may include an electronic circuit, a device including the electronic circuit, or the like.

In the present specification (including the claims), when a plurality of storage devices (memories) store data, an individual storage device among the plurality of storage devices may store only a part of the data or may store the entire data. Further, some storage devices among the plurality of storage devices may include a configuration for storing data.

The above embodiments may be summarized or expanded as follows.
(1)

An information processing device comprising:
at least one processor; and
at least one memory, wherein
the at least one processor
   acquires information about a search target from another information processing device,
   acquires a plurality of structures of a substance based on the information about the search target,
   updates the acquired plurality of structures of the substance,
   evaluates the updated plurality of structures of the substance using a neural network, and
   transmits information about a searched structure of a substance to the other information processing device based on a result of the evaluation.
(2)

The information processing device according to (1), wherein
the at least one processor updates the acquired plurality of structures of the substance using the neural network.
(3)

The information processing device according to (2), wherein
the at least one processor executes the update by converting the acquired plurality of structures of the substance to stable structures in neighborhoods.
(4)

The information processing device according to (1), wherein
the at least one processor evaluates the updated plurality of structures of the substance based on a method of multi-objective optimization.
(5)

The information processing device according to (4), wherein
the at least one processor acquires the plurality of structures of the substance based on a Pareto front.
(6)

The information processing device according to (5), wherein
the at least one processor acquires the plurality of structures of the substance using an evolutionary algorithm.
(7)

The information processing device according to any one of (1) to (6), wherein
the at least one processor repeatedly executes the acquisition, the update, and the evaluation until a predetermined condition is satisfied.
(8)

The information processing device according to (7), wherein
the at least one processor executes the acquisition, the update, and the evaluation in parallel.
(9)

The information processing device according to any one of (1) to (8), wherein
the information about the search target includes information about at least either a type of a chemical element or a composition ratio of chemical elements.
(10)

The information processing device according to (9), wherein
the information about the search target further includes information about at least one of a temperature, a pressure, a range of a convex hull, and a range of a physical property value.
(11)

The information processing device according to any one of (1) to (10), wherein
the at least one memory transmits information about convex hulls of a plurality of searched substances to the other information processing device.
(12)

The information processing device according to any one of (1) to (11), wherein
the neural network is NNP (Neural Network Potential).
(13)

The information processing device according to (12), wherein
the at least one processor evaluates the updated plurality of structures of the substance by energy.
(14)

The information processing device according to (12) or (13), wherein
the at least one memory evaluates the searched structure of the substance using a DFT (Density Functional Theory) calculation.
(15)

An information processing system comprising:
the information processing device according to any one of (1) to (14); and
at least the other information processing device.
(16)

The information processing system according to (15), wherein
the neural network is NNP (Neural Network Potential).
(17)

The information processing system according to (16), wherein
at least either the information processing device or the other information processing device evaluates the searched structure of the substance using a DFT (Density Functional Theory) calculation.
(18)

An information processing method comprising:
at least one processor
   acquiring information about a search target from another information processing device,
   acquiring a plurality of structures of a substance based on the information about the search target,
   updating the acquired plurality of structures of the substance,
   evaluating the updated plurality of structures of the substance using a neural network, and
   transmitting information about a searched structure of a substance to the other information processing device based on a result of the evaluation.
(19)

The information processing method according to (18), wherein at least a part of the information processing device according to any one of (1) to (17) is executed by the at least one processor.

While certain embodiments of the present disclosure have been described in detail above, the present disclosure is not limited to the individual embodiments described above. Various additions, changes, substitutions, partial deletions, etc. are possible to the extent that they do not deviate from the conceptual idea and purpose of the present disclosure derived from the contents specified in the claims and their equivalents. For example, when numerical values or mathematical formulas are used in the description in the above-described embodiments, they are shown for illustrative purposes only and do not limit the scope of the present disclosure. Further, the order of each operation shown in the embodiments is also an example, and does not limit the scope of the present disclosure.

1. An information processing device comprising:
at least one memory; and
at least one processor configured to:
acquire, from another information processing device, information about a search target including at least information on a chemical element;
repeatedly execute acquisition of a plurality of structures each containing the chemical element, update of the plurality of structures, and evaluation of the updated plurality of structures using a neural network, until a predetermined condition is satisfied; and
after the repeated execution, transmit information about a searched structure containing the chemical element and information about an evaluation value of the searched structure to the other information processing device;
wherein the at least one processor executes, in the repeated execution, the acquisition based on the evaluation.

2. The information processing device according to claim 1, wherein the at least one processor executes at least part of the acquisition, the update, and the evaluation in the repeated execution, in parallel.

3. The information processing device according to claim 1, wherein the at least one processor transmits, after the repeated execution, information about the searched structure and the evaluation value, about a plurality of composition ratios, to the other information processing device.

4. The information processing device according to claim 1, wherein the evaluation value includes at least one of a physical property value of the searched structure, energy of the searched structure, information on a distance between a convex hull and the energy, or information about the convex hull.

5. The information processing device according to claim 1, wherein the at least one processor transmits information on the searched structure having at least one of a distance from a convex hull falling within a predetermined value or a physical property value falling within a desired range, to the other information processing device.

6. The information processing device according to claim 1, wherein the predetermined condition includes a condition about at least one of a number of the plurality of structures for which the evaluation has been completed, time required for the search, a physical property value of the searched structure, or a number of times of the repetition.

7. The information processing device according to claim 1, wherein the at least one processor uses the neural network to update the plurality of structures obtained.

8. The information processing device according to claim 7, wherein the neural network used for the evaluation and the neural network used for the update are different neural networks.

9. The information processing device according to claim 7, wherein the at least one processor executes the update by converting the plurality of structures to stable structures in neighborhoods.

10. The information processing device according to claim 1, wherein the at least one processor evaluates the updated plurality of structures based on a method of multi-objective optimization.

11. The information processing device according to claim 1, wherein the at least one processor acquires the plurality of structures based on a Pareto front.

12. The information processing device according to claim 1, wherein the at least one processor acquires the plurality of structures using an evolutionary algorithm.

13. The information processing device according to claim 1, wherein the information about the search target further includes information about a composition ratio of chemical elements.

14. The information processing device according to claim 13, wherein the information about the search target further includes information about at least one of a temperature, a pressure, a range of a convex hull, or a range of a physical property value.

15. The information processing device according to claim 1, wherein the at least one processor transmits information about convex hulls of a plurality of searched substances to the other information processing device.

16. The information processing device according to claim 1, wherein the neural network is NNP (Neural Network Potential).

17. The information processing device according to claim 16, wherein the at least one processor evaluates the updated plurality of structures by energy.

18. The information processing device according to claim 16, wherein the at least one processor evaluates the searched structure using a DFT (Density Functional Theory) calculation.

19. The information processing device according to claim 1, wherein the search target is a crystal structure.

20. The information processing device according to claim 1 providing a service in a form of a Saas (Software as a Service) to the other information processing device.

21. An information processing system comprising:
a first information processing device; and
a second information processing device,
wherein the first information processing device is configured to:
acquire, from the second information processing device, information about a search target including at least information on a chemical element;
repeatedly execute acquisition of a plurality of structures each containing the chemical element, update of the plurality of structures, and evaluation of the updated plurality of structures using a neural network, until a predetermined condition is satisfied; and
after the repeated execution, transmit information about a searched structure containing the chemical element and information about an evaluation value of the searched structure to the second information processing device;
wherein the first information processing device executes, in the repeated execution, the acquisition based on the evaluation.

22. An information processing method comprising:
acquiring, by at least one processor, from another information processing device, information about a search target including at least information on a chemical element;
repeatedly executing, by the at least one processor, acquisition of a plurality of structures each containing the chemical element, update of the plurality of structures, and evaluation of the updated plurality of structures using a neural network, until a predetermined condition is satisfied; and
after the repeated execution, transmitting, by the at least one processor, information about a searched structure containing the chemical element and information about an evaluation value of the searched structure to the other information processing device;
wherein the at least one processor executes, in the repeated execution, the acquisition based on the evaluation.

23. A non-transitory computer readable medium storing program that when executed by at least one processor of an information processing device, performs an information processing method comprising:
acquiring, from another information processing device, information about a search target including at least information on a chemical element;
repeatedly executing acquisition of a plurality of structures each containing the chemical element, update of the plurality of structures, and evaluation of the updated plurality of structures using a neural network, until a predetermined condition is satisfied; and
after the repeated execution, transmitting information about a searched structure containing the chemical element and information about an evaluation value of the searched structure to the other information processing device
wherein the at least one processor executes, in the repeated execution, the acquisition based on the evaluation.
